(19) United States
(12) Patent Application Publication
(10) Pub. No.: US 2025/0260264 A1
(43) Pub. Date: Aug. 14, 2025

(54) WIRELESS POWER TRANSMISSION APPARATUS AND IMAGING DEVICE COMPRISING SAME

(71) Applicants: SHANGHAI UNITED IMAGING HEALTHCARE CO., LTD., Shanghai (CN); HUAZHONG UNIVERSITY OF SCIENCE AND TECHNOLOGY, Wuhan (CN)

(72) Inventors: Yaoqiang DUAN, Wuhan (CN); Zhi CHEN, Wuhan (CN); Tieshan ZHANG, Shanghai (CN); Yajun MA, Wuhan (CN); Xu CHU, Shanghai (CN); Zicheng LIU, Wuhan (CN); Longyuan FAN, Wuhan (CN); Dong JIANG, Wuhan (CN)

(73) Assignees: SHANGHAI UNITED IMAGING HEALTHCARE CO., LTD., Shanghai (CN); HUAZHONG UNIVERSITY OF SCIENCE AND TECHNOLOGY, Wuhan (CN)

(21) Appl. No.: 19/192,300

(22) Filed: Apr. 28, 2025

Related U.S. Application Data
(63) Continuation of application No. PCT/CN2023/127725, filed on Oct. 30, 2023.

(30) Foreign Application Priority Data
Oct. 28, 2022 (CN) 202211338877.2

(51) Int. Cl. H02J 50/00; A61B 6/00; H02J 50/10

(52) U.S. Cl. CPC H02J 50/005; A61B 6/56; H02J 50/10; H02J 2310/23

(57) ABSTRACT

The present disclosure discloses a wireless power transmission apparatus and an imaging device comprising the same. The wireless power transmission apparatus comprises: a first annular structure, provided with a first magnetic core assembly and a first framework, wherein the first magnetic core assembly is provided on a circumference of the first annular structure, the first framework is wound with a first winding, and the first winding passes through a window of the magnetic core in the first magnetic core assembly; and a second annular structure, provided with a second framework, wherein the second framework is wound with a second winding, and the second winding passes through the window of the magnetic core in the first magnetic core assembly; and the first winding is used for receiving an alternating voltage and transmitting the alternating voltage to the second winding.

### CROSS-REFERENCE TO RELATED APPLICATIONS

This application is a continuation of International Patent Application No. PCT/CN2023/127725, filed on Oct. 30, 2023, which claims the priority of Chinese Patent Application No. CN202211338877.2 filed on Oct. 28, 2022, the content of which is incorporated herein by reference in its entirety.

### TECHNICAL FIELD

The present disclosure relates to the field of medical devices, and in particular, to a wireless power transmission apparatus and an imaging device comprising same.

### BACKGROUND

In recent years, great development has been made in X-ray Computed Tomography (CT) in both basic technologies and new clinical applications, and it has become one of the most exciting diagnostic methods in the field of medical images. The power transmission link of a traditional CT device is as shown in FIG. 1, and it mainly includes a Power Distribution Unit (PDU), an electric brush, a slip ring, a High Voltage Generator (HVG), an X-ray tube assembly, a detector assembly and other auxiliary control system units. The PDU unit is located on a stationary side and is internally provided with a power frequency isolation transformer and a protection control unit. The HVG, the X-ray tube assembly and the detector are usually fixed on a rotating gantry and rotate with the gantry. The slip ring is arranged on the rotating gantry, and power output by the PDU is transmitted to the rotating gantry through contact friction between the electric brush arranged on the stationary side and the slip ring on the rotating gantry, so as to supply power to the HVG, the detector and other components. The HVG converts the input low voltage to the high DC voltage (up to 140 kV), which is applied between an anode and a cathode of the X-ray tube assembly to generate a high voltage electric field between the anode and the cathode of the X-ray tube assembly. Meanwhile, the HVG provides current for a filament at the cathode of the X-ray tube assembly, so that the filament is heated to generate free electrons at the cathode, and under the control of the high voltage electric field, the electrons impact an anode target at a high speed to generate X-rays. In addition, the HVG also provides power to drive the motor at the anode of the X-ray tube assembly, so that the anode target rotates at a high speed, thus facilitating heat dissipation of the anode target.

However, in the power transmission link of a traditional CT device, the power of the CT device is transmitted by the friction between the electric brush and the slip ring, which increases the maintenance cost of the CT device and reduces its reliability.

In addition, the HVG, the X-ray tube assembly and the detector are all located on the rotating gantry. The HVG usually includes an inverter and a high-voltage tank and thus occupies a large part of the space on the rotating gantry. The diameter and volume of the rotating gantry increase along with the increase of the power of the CT device. Moreover, in order to keep the dynamic balance of the rotating gantry resulted by the large weight of the HVG, a corresponding counterweight is added to the rotating gantry to keep the balance of the gantry, which greatly increases the load on the rotating gantry. The maximum rotating speed of the rotating gantry is limited by the large diameter and high load of the gantry. Besides, when a traditional CT device is in scanning operation with higher instantaneous power (which is usually tens of kilowatts or even hundreds of kilowatts), the power grid and the PDU also need to fulfill the high instantaneous power. Thus, it is required that the power distribution capacity of a mounting site should be greater than the instantaneous power of the CT device. This puts a high requirement on the power distribution capacity of the equipment mounting site.

### CONTENT OF THE PRESENT INVENTION

The present disclosure provides a wireless power transmission apparatus and an imaging device comprising the same to overcome the drawbacks in the prior art that the power of a CT device being transmitted by the friction between the electric brush and the slip ring increases the maintenance cost of the CT device and reduces its reliability.

The present disclosure solves the above technical problems through the following technical solutions:

A first aspect of the present disclosure provides a wireless power transmission apparatus, which comprises: a first annular structure, provided with a first magnetic core assembly and a first framework, wherein the first magnetic core assembly is arranged on a circumference of the first annular structure, the first magnetic core assembly comprises at least one magnetic core, the first framework is wound with a first winding, and the first winding passes through a window of the magnetic core in the first magnetic core assembly; and a second annular structure, provided with a second framework, wherein the second framework is wound with a second winding, and the second winding passes through the window of the magnetic core in the first magnetic core assembly; wherein a gap is present between the first winding and the second winding, the second annular structure is rotatable relative to the first annular structure, and the first winding is used for receiving an alternating voltage and transmitting the alternating voltage to the second winding.

Optionally, the magnetic core in the first magnetic core assembly has a magnetic core gap, a size of the magnetic core gap meets a requirement for the second framework to pass through the magnetic core gap, and the second framework passes through the magnetic core gap and is fixed on the second annular structure.

Optionally, the first magnetic core assembly is arranged on an inner side and/or an outer side of the circumference of the first annular structure.

Optionally, the magnetic cores in the first magnetic core assembly are uniformly arranged on the circumference of the first annular structure.

Optionally, the first framework and/or the second framework is a closed annular structure.

Optionally, the first frameworks are a plurality of different structures, and/or the second frameworks are a plurality of different structures.

Optionally, the circumference of the first annular structure is also provided with a second magnetic core assembly, the second magnetic core assembly comprises at least one magnetic core, the first annular structure is further provided with a third framework, the third framework is wound with a third winding, and the third winding passes through a window of the magnetic core in the second magnetic core assembly; the second annular structure is further provided with a fourth framework, the fourth framework is wound with a fourth winding, and the fourth winding passes through the window of the magnetic core in the second magnetic core assembly; wherein a gap is present between the third winding and the fourth winding, and the third winding is used for receiving an alternating voltage and transmitting the alternating voltage to the fourth winding.

Optionally, the magnetic core in the second magnetic core assembly has a magnetic core gap, a size of the magnetic core gap meets a requirement for the fourth framework to pass through the magnetic core gap, and the fourth framework passes through the magnetic core gap and is fixed on the second annular structure.

Optionally, the magnetic cores in the second magnetic core assembly are uniformly arranged on the circumference of the first annular structure.

Optionally, the second magnetic core assembly and the first magnetic core assembly are arranged side by side on the circumference of the first annular structure, the third framework and the first framework are arranged side by side on the first annular structure, and the fourth framework and the second framework are arranged side by side on the second annular structure.

Optionally, the third framework and the first framework are arranged side by side on the inner side or the outer side of the first annular structure.

Optionally, the third frameworks and the first frameworks are in one-to-one correspondence in position or are arranged in an alternating manner.

Optionally, the fourth frameworks and the second frameworks are in one-to-one correspondence in position or are arranged in an alternating manner.

Optionally, the quantity of the third frameworks and the quantity of the first frameworks are different, and/or the quantity of the fourth frameworks and the quantity of the second frameworks are different

A second aspect of the present disclosure provides an imaging device, which comprises a gantry and the wireless power transmission apparatus described in the first aspect. The first annular structure of the wireless power transmission apparatus is located on a stationary side of the gantry, and the second annular structure of the wireless power transmission apparatus is located on a rotating side of the gantry.

Optionally, the imaging device further comprises a power distribution unit, a step-up transformer module, a rectifier-filter module and an X-ray tube assembly, the power distribution unit is located on the stationary side of the gantry and comprises a first rectifier circuit and a first inverter circuit, and the step-up transformer module, the rectifier-filter module and the X-ray tube assembly are all located on the rotating side of the gantry;
an output end of the first rectifier circuit is connected to an input end of the first inverter circuit, an output end of the first inverter circuit is connected to the first winding in the wireless power transmission apparatus, the second winding in the wireless power transmission apparatus is connected to an input end of the step-up transformer module, an output end of the step-up transformer module is connected to an input end of the rectifier-filter module, and an output end of the rectifier-filter module is connected to the X-ray tube assembly; the first rectifier circuit is used for receiving an alternating voltage provided by a power grid, rectifying the alternating voltage and outputting a DC voltage; the first inverter circuit is used for converting the DC voltage output by the first rectifier circuit into an alternating voltage and transmitting the alternating voltage to the step-up transformer module through the first winding and the second winding in the wireless power transmission apparatus; the step-up transformer module is used for receiving the alternating voltage transmitted by the wireless power transmission apparatus and stepping up the alternating voltage; and the rectifier-filter module is used for rectifying and filtering the stepped-up alternating voltage and applying an obtained DC voltage to the X-ray tube assembly.

Optionally, the step-up transformer module, the rectifier-filter module and the X-ray tube assembly are located in a same housing, and the housing is filled with an insulating medium.

Optionally, the power distribution unit further comprises an energy storage unit, and the energy storage unit is connected to the output end of the first rectifier circuit.

Optionally, the first rectifier circuit is an uncontrollable rectifier circuit, and the power distribution unit further comprises a bidirectional direct current converter connected in series between the first rectifier circuit and the energy storage unit.

Optionally, the power distribution unit further comprises a second inverter circuit, the imaging device further comprises a second rectifier circuit, a filament transformer and a filament control unit, the second rectifier circuit, the filament transformer and the filament control unit are all located on the rotating side of the gantry, the filament transformer comprises a primary winding and a secondary winding, and the X-ray tube assembly comprises a filament;
the output end of the first rectifier circuit is also connected to an input end of the second inverter circuit, an output end of the second inverter circuit is connected to the third winding in the wireless power transmission apparatus, the fourth winding in the wireless power transmission apparatus is connected to an input end of the second rectifier circuit, an output end of the second rectifier circuit is connected to an input end of the filament control unit, an output end of the filament control unit is connected to the primary winding of the filament transformer, and the secondary winding of the filament transformer is connected to the filament; the second inverter circuit is used for converting the DC voltage output by the first rectifier circuit into an alternating voltage and transmitting the alternating voltage to the second rectifier circuit through the third winding and the fourth winding in the wireless power transmission apparatus; the second rectifier circuit is used for receiving the alternating voltage transmitted by the wireless power transmission apparatus and rectifying the alternating voltage to obtain a DC voltage; and the filament control unit is used for converting the DC voltage output by the second rectifier circuit into an alternating voltage and loading the alternating voltage to the primary winding of the filament transformer.

On the basis of the general knowledge in the art, the optional conditions described above can be combined arbitrarily to obtain the preferred embodiments of the present disclosure.

The present disclosure has the following positive and progressive effect: in the wireless power transmission apparatus provided by the present disclosure, the first magnetic core assembly is fixed on the first annular structure on a stationary side, which can reduce the weight of the second annular structure on a rotating side and can also improve the safety of a magnetic core structure.

In addition, applying the wireless power transmission apparatus provided by the present disclosure to a CT device can reduce the weight of the rotating side of the gantry, which can improve the rotating speed of the gantry and thereby improve the time resolution of scanning of the CT device.

### BRIEF DESCRIPTION OF THE DRAWINGS

FIG. 1 is a schematic diagram of a power transmission link of a CT device in the prior art.

FIG. 2 is a schematic diagram of a three-dimensional structure of a wireless power transmission apparatus provided by Embodiment 1 of the present disclosure.

FIG. 3 is a cross-sectional view of FIG. 2.

FIG. 4 is a schematic cross-sectional view of another wireless power transmission apparatus provided by Embodiment 1 of the present disclosure.

FIG. 5 is a schematic cross-sectional view of another wireless power transmission apparatus provided by Embodiment 1 of the present disclosure.

FIG. 6 is a schematic cross-sectional view of another wireless power transmission apparatus provided by Embodiment 1 of the present disclosure.

FIG. 7 is a schematic cross-sectional view of another wireless power transmission apparatus provided by Embodiment 1 of the present disclosure.

FIG. 8 is a structural block diagram of an imaging device provided by Embodiment 2 of the present disclosure.

FIG. 9 is a circuit connection diagram of a filament control unit and a filament transformer provided by Embodiment 2 of the present disclosure.

FIG. 10 is a circuit connection diagram of an anode driver and a motor provided by Embodiment 2 of the present disclosure.

FIG. 11 is a schematic diagram of a circuit structure of an imaging device provided by Embodiment 2 of the present disclosure.

FIG. 12 is a schematic diagram of a circuit structure of another imaging device provided by Embodiment 2 of the present disclosure.

FIG. 13 is a structural block diagram of an imaging device provided by Embodiment 2 of the present disclosure.

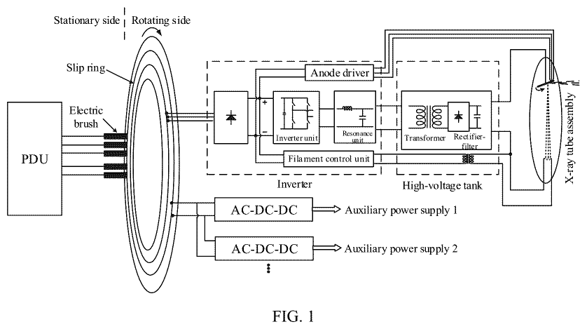

### DETAILED DESCRIPTION OF THE PREFERRED EMBODIMENT

The present disclosure is further illustrated by the following embodiments, which are not intended to limit the present disclosure.

### Embodiment 1

This embodiment provides a wireless power transmission apparatus, which comprises a first annular structure and a second annular structure. A first magnetic core assembly is arranged on a circumference of the first annular structure, the first magnetic core assembly comprises at least one magnetic core, the first annular structure is provided with a first framework, the first framework is wound with a first winding, and the first winding passes through a window of the magnetic core in the first magnetic core assembly. The second annular structure is provided with a second framework, the second framework is wound with a second winding, and the second winding passes through the window of the magnetic core in the first magnetic core assembly. Where the window of the magnetic core refers to an open pore used for accommodating the frameworks and the windings.

Where a gap is present between the first winding and the second winding, the second annular structure is rotatable relative to the first annular structure, and the first winding is used for receiving an alternating voltage and transmitting the alternating voltage to the second winding. In a specific implementation, the first annular structure is used for being fixedly connected to a gantry of an imaging device and is located on a stationary side of the gantry, and the second annular structure is located on a rotating side of the gantry. During operation of the wireless power transmission apparatus, the second annular structure rotates as the gantry rotates, and the first annular structure and the second annular structure rotate relative to each other.

In a specific implementation, a plurality of first frameworks may be arranged on the first annular structure, and the first winding is wound on each first framework. An annular first framework may also be arranged on the first annular structure, and the first winding is wound on the first framework. Where the first framework and the first annular structure may be fixedly connected or detachably connected.

Similarly, a plurality of second frameworks may be arranged on the second annular structure, and the second winding is wound on each second framework. An annular second framework may also be arranged on the second annular structure, and the second winding is wound on the second framework, wherein the second winding is an annular winding surrounding the second annular structure. Where the second framework and the second annular structure may be fixedly connected or detachably connected.

In this embodiment, a certain gap is reserved between the first winding on the stationary side and the second winding on the rotating side, so that it can be ensured that the first winding and the second winding will not be in contact with each other during relative rotation. The first winding and the second winding are magnetically coupled by the first magnetic core assembly to transfer electric energy received by the first winding to the second winding wirelessly, thus realizing wireless transmission of power. Therefore, the power transmission apparatus provided by this embodiment is a wireless power transmission apparatus.

In the wireless power transmission apparatus provided by this embodiment, the first magnetic core assembly is fixed on the first annular structure on the stationary side, which can reduce the weight of the second annular structure on the rotating side, and can also improve the safety of a magnetic core structure since the magnetic cores do not need to rotate during operation of the wireless power transmission apparatus.

In addition, applying the wireless power transmission apparatus provided by this embodiment to a CT device can reduce the weight of the rotating side of the gantry, which can improve the rotating speed of the gantry and thereby improve the time resolution of scanning of the CT device.

In an optional implementation, the magnetic core in the first magnetic core assembly has a magnetic core gap, and a size of the magnetic core gap meets a requirement for the second framework to pass through the magnetic core gap; the second framework passes through the magnetic core gap and is fixed on the second annular structure. In this implementation, the first winding and the second winding can be magnetically coupled by the first magnetic core assembly and the magnetic core gaps of the magnetic cores in the first magnetic core assembly, so as to transmit the electric energy received by the first winding to the second winding. Where when the second annular structure rotates, any second framework on the second annular structure can pass through any magnetic core gap in the first magnetic core assembly. Specifically, the structure of the magnetic core may be UU type, UY type and the like, which will not be limited specifically.

Where the first magnetic core assembly is arranged on the circumference of the first annular structure. Specifically, the first magnetic core assembly may be arranged on an inner side or an outer side of the first annular structure, or may be arranged on both the inner side and the outer side of the first annular structure.

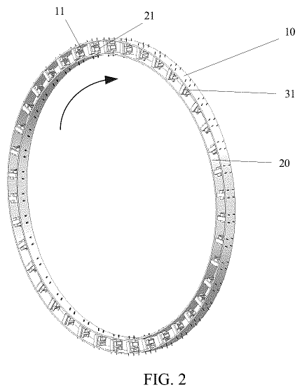

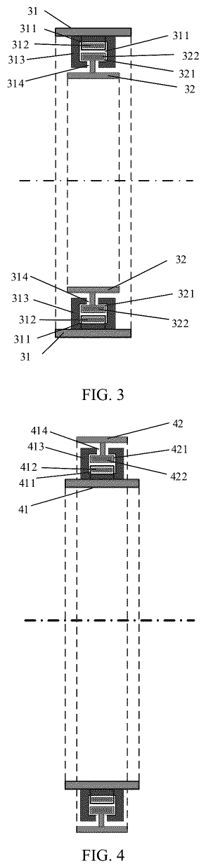

FIG. 2 is a diagram of a three-dimensional structure of a wireless power transmission apparatus, and FIG. 3 is a cross-sectional view of the wireless power transmission apparatus in FIG. 2. The wireless power transmission apparatus as shown in FIG. 2 and FIG. 3 comprises a first annular structure 31 and a second annular structure 32. The first annular structure 31 is provided with a plurality of first frameworks 311. The first frameworks 311 are wound with first windings 312. The second annular structure 32 is provided with a plurality of second frameworks 321. The second framework 321 has a T-shaped cross section and is wound with a second winding 322. A first magnetic core assembly is arranged on an inner side of the first annular structure 31, and the first magnetic core assembly comprises a plurality of magnetic cores 313. The first frameworks 311 and the second frameworks 321 all pass through windows of the magnetic cores 313. One end of the magnetic core 313 that is far away from the first annular structure 31 has a magnetic core gap 314. The second framework 321 passes through the magnetic core gap 314 and is fixed on the second annular structure 32. In the example shown in FIG. 3, the second annular structure 32 is an annular structure smaller than the first annular structure 31, and the second annular structure 32 and the first annular structure 31 correspond to the same circle center.

The wireless power transmission apparatus shown in FIG. 4 comprises a first annular structure 41 and a second annular structure 42. The first annular structure 41 is provided with a plurality of first frameworks 411. The first frameworks 411 are wound with first windings 412. The second annular structure 42 is provided with a plurality of second frameworks 421. The second framework 421 has a T-shaped cross section and is wound with a second winding 422. A first magnetic core assembly is arranged on an outer side of the first annular structure 41, and the first magnetic core assembly comprises a plurality of magnetic cores 413. The first frameworks 411 and the second frameworks 421 all pass through windows of the magnetic cores 413. One end of the magnetic core 413 that is far away from the first annular structure 41 has a magnetic core gap 414. The second framework 421 passes through the magnetic core gap 414 and is fixed on the second annular structure 42. In the example shown in FIG. 4, the second annular structure 42 is an annular structure larger than the first annular structure 41, and the second annular structure 42 and the first annular structure 41 correspond to the same circle center.

The wireless power transmission apparatus shown in FIG. 5 comprises a first annular structure 51, a second annular structure 52 and a second annular structure 53. The first annular structure 51 is provided with a plurality of first frameworks 511 and a plurality of first frameworks 512. The first frameworks 511 are wound with first windings 5111, and the first frameworks 512 are wound with first windings 5121. The second annular structure 52 is provided with a plurality of second frameworks 521. The second framework 521 has a T-shaped cross section and is wound with a second winding 522. The second annular structure 53 is provided with a plurality of second frameworks 531. The second framework 531 has a T-shaped cross section and is wound with a second winding 532. A first magnetic core assembly arranged on an inner side of the first annular structure 51 comprises a plurality of magnetic cores 513. The first frameworks 511 and the second frameworks 521 all pass through windows of the magnetic cores 513. One end of the magnetic core 513 that is far away from the first annular structure 51 has a magnetic core gap 5131. The second framework 521 passes through the magnetic core gap 5131 and is fixed on the second annular structure 52. A first magnetic core assembly arranged on an outer side of the first annular structure 51 comprises a plurality of magnetic cores 514. The first frameworks 512 and the second frameworks 531 all pass through windows of the magnetic cores 514. One end of the magnetic core 514 that is far away from the first annular structure 51 has a magnetic core gap 5141. The second framework 531 passes through the magnetic core gap 5141 and is fixed on the second annular structure 53. In the example shown in FIG. 5, the first annular structure 51 is located between the second annular structure 52 and the second annular structure 53, and the first annular structure 51, the second annular structure 52 and the second annular structure 53 all correspond to the same circle center.

In a specific implementation, the magnetic cores in the first magnetic core assembly may be non-uniformly arranged on a circumference of the first annular structure.

Preferably, the magnetic cores in the first magnetic core assembly are uniformly arranged on the circumference of the first annular structure. In this implementation, since the magnetic cores in the first magnetic core assembly are uniformly arranged around the first annular structure, the first framework, the first winding and the second framework corresponding to the same magnetic core in the first magnetic core assembly are all correspondingly uniformly arranged, making the stress on the wireless power transmission apparatus uniform. Thus, the stability of the wireless power transmission apparatus during operation can be improved.

In a specific implementation, the first framework described above may be a closed annular structure, or may be a plurality of different structures, e.g., a plurality of structures with different lengths and/or shapes. Similarly, the second framework described above may be a closed annular structure, or may be a plurality of different structures, e.g., a plurality of structures with different lengths and/or shapes.

In an optional implementation, a second magnetic core assembly is also arranged on the circumference of the first annular structure. The second magnetic core assembly comprises at least one magnetic core. The first annular structure is provided with a third framework. The third framework is wound with a third winding. The third winding passes through a window of the magnetic core in the second magnetic core assembly. The second annular structure is also provided with a fourth framework, and the fourth framework is wound with a fourth winding. The fourth winding passes through the window of the magnetic core in the second magnetic core assembly. Where a gap is present between the third winding and the fourth winding. The third winding is used for receiving an alternating voltage and transmitting the alternating voltage to the fourth winding.

Where the third framework and the first annular structure may be fixedly connected or detachably connected. There may be one third framework or a plurality of third frameworks. The fourth framework and the second annular structure may be fixedly connected or detachably connected. There may be one fourth framework or a plurality of fourth frameworks.

Where the third framework and the first framework can be arranged side by side on the first annular structure, the second magnetic core assembly and the first magnetic core assembly can be arranged side by side on the circumference of the first annular structure, and the fourth framework and the second framework can be arranged side by side on the second annular structure, so that two transmission channels of the wireless power transmission apparatus can be formed. Specifically, the first magnetic core assembly and the second magnetic core assembly can be arranged side by side on the inner side of the first annular structure, and the third framework and the first framework can be arranged side by side on the inner side of the first annular structure. Or, the first magnetic core assembly and the second magnetic core assembly can be arranged side by side on the outer side of the first annular structure, and the third framework and the first framework can be arranged side by side on the outer side of the first annular structure. Where the magnetic cores in the first magnetic core assembly and the magnetic cores in the second magnetic core assembly may be different, specifically in quantity, model type, structure and the like. The third frameworks and the first frameworks may be in one-to-one correspondence in position or may be arranged in an alternating manner, and the quantity of the third frameworks and that of the first frameworks may be the same or different. The fourth frameworks and the second frameworks may be in one-to-one correspondence in position or may be arranged in an alternating manner. The quantity of the fourth frameworks and that of the second frameworks may be the same or different. In a specific implementation, the first annular structure and the second annular structure both have a certain width. A whole composed of the plurality of third frameworks and a whole composed of the plurality of first frameworks are arranged side by side on the first annular structure, and a whole composed of the plurality of fourth frameworks and a whole composed of the plurality of second frameworks are arranged side by side on the second annular structure.

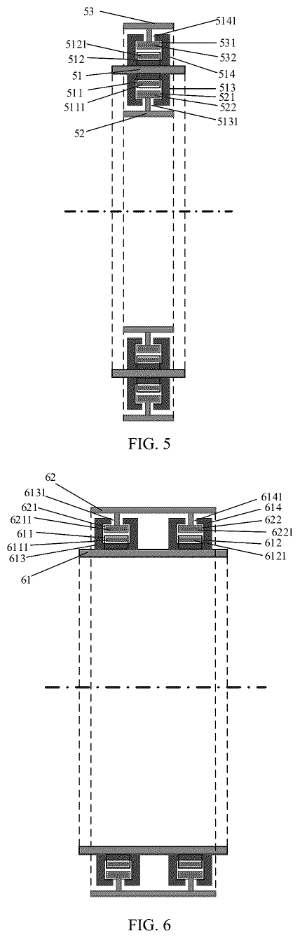

The wireless power transmission apparatus shown in FIG. 6 comprises a first annular structure 61 and a second annular structure 62. A plurality of first frameworks 611 and a plurality of third frameworks 612 are arranged on an outer side of the first annular structure 61. The first frameworks 611 and the third frameworks 612 are arranged side by side. The first frameworks 611 are wound with first windings 6111, and the third frameworks 612 are wound with third windings 6121. A plurality of second frameworks 621 and a plurality of fourth frameworks 622 are arranged on the second annular structure 62. The second frameworks 621 and the fourth frameworks 622 are arranged side by side. The second frameworks 621 are wound with second windings 6211, and the fourth frameworks 622 are wound with fourth windings 6221. A first magnetic core assembly and a second magnetic core assembly are arranged side by side on the outer side of the first annular structure 61. The first magnetic core assembly comprises a plurality of magnetic cores 613. The second magnetic core assembly comprises a plurality of magnetic cores 614. The first frameworks 611 and the second frameworks 621 all pass through windows of the magnetic cores 613. One end of the magnetic core 613 that is far away from the first annular structure 61 has a magnetic core gap 6131. The second framework 621 passes through the magnetic core gap 6131 and is fixed on the second annular structure 62. The third frameworks 612 and the fourth frameworks 622 all pass through windows of the magnetic cores 614. One end of the magnetic core 614 that is far away from the first annular structure 61 has a magnetic core gap 6141. The fourth framework 622 passes through the magnetic core gap 6141 and is fixed on the second annular structure 62. In the example shown in FIG. 6, the second annular structure 62 is an annular structure larger than the first annular structure 61, and the second annular structure 62 and the first annular structure 61 correspond to the same circle center.

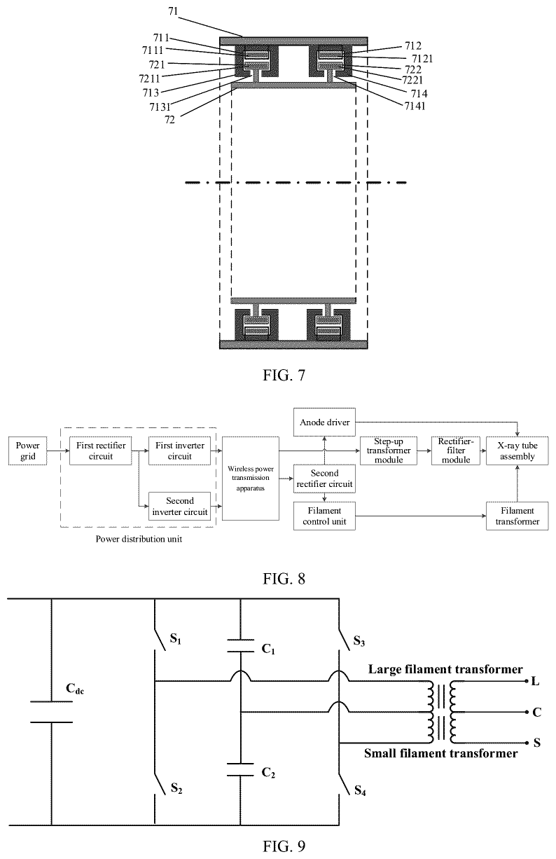

The wireless power transmission apparatus shown in FIG. 7 comprises a first annular structure 71 and a second annular structure 72. A plurality of first frameworks 711 and a plurality of third frameworks 712 are arranged on an inner side of the first annular structure 71. The first frameworks 711 and the third frameworks 712 are arranged side by side. The first frameworks 711 are wound with first windings 7111, and the third frameworks 712 are wound with third windings 7121. A plurality of second frameworks 721 and a plurality of fourth frameworks 722 are arranged on the second annular structure 72. The second frameworks 721 and the fourth frameworks 722 are arranged side by side. The second frameworks 721 are wound with second windings 7211, and the fourth frameworks 722 are wound with fourth windings 7221. A first magnetic core assembly and a second magnetic core assembly are arranged side by side on the inner side of the first annular structure 71. The first magnetic core assembly comprises a plurality of magnetic cores 713. The second magnetic core assembly comprises a plurality of magnetic cores 714. The first frameworks 711 and the second frameworks 721 all pass through windows of the magnetic cores 713. One end of the magnetic core 713 that is far away from the first annular structure 71 has a magnetic core gap 7131. The second framework 721 passes through the magnetic core gap 7131 and is fixed on the second annular structure 72. The third frameworks 712 and the fourth frameworks 722 all pass through windows of the magnetic cores 714. One end of the magnetic core 714 that is far away from the first annular structure 71 has a magnetic core gap 7141. The fourth framework 722 passes through the magnetic core gap 7141 and is fixed on the second annular structure 72. In the example shown in FIG. 7, the second annular structure 72 is an annular structure smaller than the first annular structure 71, and the second annular structure 72 and the first annular structure 71 correspond to the same circle center.

In this implementation, the first magnetic core assembly and the second magnetic core assembly are arranged on the circumference of the first annular structure, the first annular structure is provided with the first frameworks and the third frameworks, and the second annular structure is provided with the second frameworks and the fourth frameworks. The first windings wound on the first frameworks and the second windings wound on the second frameworks can form a first transmission channel of the wireless power transmission apparatus. Specifically, an alternating voltage received by the first windings is transmitted to the second windings. The third windings wound on the third frameworks and the fourth windings wound on the fourth frameworks can form a second transmission channel of the wireless power transmission apparatus. Specifically, an alternating voltage received by the third windings is transmitted to the fourth windings.

In an optional implementation, the magnetic core in the second magnetic core assembly has a magnetic core gap, and a size of the magnetic core gap meets a requirement for the fourth framework to pass through the magnetic core gap; the fourth framework passes through the magnetic core gap and is fixed on the second annular structure. In this implementation, the third winding and the fourth winding can be magnetically coupled by the second magnetic core assembly and the magnetic core gaps of the magnetic cores in the second magnetic core assembly, so as to transmit the electric energy received by the third winding to the fourth winding. Where when the second annular structure rotates, any fourth framework on the second annular structure can pass through any magnetic core gap in the second magnetic core assembly. Specifically, the structure of the magnetic core may be UU type, UY type and the like, which will not be limited specifically.

In a specific implementation, the magnetic cores in the second magnetic core assembly may be non-uniformly arranged on the circumference of the first annular structure. Preferably, all the magnetic cores in the second magnetic core assembly are uniformly arranged on the circumference of the first annular structure. In this implementation, since all the magnetic cores in the second magnetic core assembly are uniformly arranged around the first annular structure, the third framework, the third winding and the fourth framework corresponding to the same magnetic core in the second magnetic core assembly are all correspondingly uniformly arranged, making the stress on the wireless power transmission apparatus uniform. Thus, the stability of the wireless power transmission apparatus during operation is improved.

### Embodiment 2

This embodiment provides an imaging device, which comprises a gantry and the wireless power transmission apparatus described in Embodiment 1. The first annular structure of the wireless power transmission apparatus is located on a stationary side of the gantry, and the second annular structure of the wireless power transmission apparatus is located on a rotating side of the gantry. Where the imaging device may be an X-ray imaging device, and may specifically be a CT device and a combined imaging device of CT and other modalities, such as a PET-CT combined imaging device of CT and positron emission computed tomography (PET).

In an optional implementation, as shown in FIG. 8, the imaging device further comprises a power distribution unit, a step-up transformer module, a rectifier-filter module and an X-ray tube assembly. The power distribution unit is located on the stationary side of the gantry and comprises a first rectifier circuit and a first inverter circuit, and the step-up transformer module, the rectifier-filter module and the X-ray tube assembly are all located on the rotating side of the gantry.

An output end of the first rectifier circuit is connected to an input end of the first inverter circuit, an output end of the first inverter circuit is connected to the first winding in the wireless power transmission apparatus, the second winding in the wireless power transmission apparatus is connected to an input end of the step-up transformer module, an output end of the step-up transformer module is connected to an input end of the rectifier-filter module, and an output end of the rectifier-filter module is connected to the X-ray tube assembly.

The first rectifier circuit is used for receiving an alternating voltage provided by a power grid, rectifying the alternating voltage and outputting a DC voltage. The first inverter circuit is used for converting the DC voltage output by the first rectifier circuit into an alternating voltage and transmitting the alternating voltage to the step-up transformer module through the first winding and the second winding in the wireless power transmission apparatus. The step-up transformer module is used for receiving the alternating voltage transmitted by the wireless power transmission apparatus and stepping up the alternating voltage. The rectifier-filter module is used for rectifying and filtering the stepped-up alternating voltage and applying an obtained DC voltage to the X-ray tube assembly.

In an optional implementation, as shown in FIG. 8, the power distribution unit further comprises a second inverter circuit, and the imaging device further comprises a second rectifier circuit, a filament transformer and a filament control unit. The second rectifier circuit, the filament transformer and the filament control unit are all located on the rotating side of the gantry, the filament transformer comprises a primary winding and a secondary winding, and the X-ray tube assembly comprises at least one filament.

The output end of the first rectifier circuit is also connected to an input end of the second inverter circuit, an output end of the second inverter circuit is connected to the third winding in the wireless power transmission apparatus, the fourth winding in the wireless power transmission apparatus is connected to an input end of the second rectifier circuit, an output end of the second rectifier circuit is connected to an input end of the filament control unit, an output end of the filament control unit is connected to the primary winding of the filament transformer, and the secondary winding of the filament transformer is connected to the filament.

The second inverter circuit is used for converting the DC voltage output by the first rectifier circuit into an alternating voltage and transmitting the alternating voltage to the second rectifier circuit through the third winding and the fourth winding in the wireless power transmission apparatus. The second rectifier circuit is used for receiving the alternating voltage transmitted by the wireless power transmission apparatus and rectifying the alternating voltage to obtain a DC voltage. The filament control unit is used for converting the DC voltage output by the second rectifier circuit into an alternating voltage and loading the alternating voltage to the primary winding of the filament transformer.

In this implementation, the wireless power transmission apparatus comprises a first transmission channel and a second transmission channel, wherein the first transmission channel is composed of a first winding and a second winding, and the second transmission channel is composed of a third winding and a fourth winding. The first rectifier circuit rectifies the alternating voltage provided by the power grid into the DC voltage Udc. In one aspect, a high-frequency alternating voltage obtained after the DC voltage Udc passes through the first inverter circuit is transmitted to the rotating side of the gantry through the first transmission channel of the wireless power transmission apparatus, so as to supply power to an X-ray monoblock assembly; in another aspect, a high-frequency alternating voltage obtained after the DC voltage Udc passes through the second inverter circuit is transmitted to the rotating side of the gantry through the second transmission channel of the wireless power transmission apparatus, so as to supply power to the filament control unit and an anode driver.

In specific application of the imaging device, the first inverter circuit can be controlled in different ways according to different scanning protocols to convert the DC voltage Udc into the alternating voltage corresponding to the scanning protocol, which is then transmitted by the first transmission channel in the wireless power transmission apparatus and subjected to voltage conversion by the step-up transformer module and the rectifier-filter module, so that the voltage corresponding to the scanning protocol can be generated at an anode and an cathode of the X-ray tube assembly and the specific parts of a patient can be scanned.

In this implementation, a voltage output by the secondary winding of the filament transformer is used for power supply to the filament in the X-ray tube assembly, so that the filament is heated to generate electrons at the cathode. The electrons impact an anode target at a high speed to generate X-rays under the control of a high voltage electric field. FIG. 9 is a schematic circuit connection diagram of a filament control unit and a filament transformer. In the example shown in FIG. 9, the X-ray tube assembly comprises a large filament and a small filament. The filament transformer comprises a large filament transformer and a small filament transformer. The filament control unit uses dual half-bridge circuits. Output ends of the dual half-bridge circuits are respectively connected to the large filament transformer and the small filament transformer. A voltage output by the large filament transformer is supplied to the large filament, and a voltage output by the small filament transformer is supplied to the small filament.

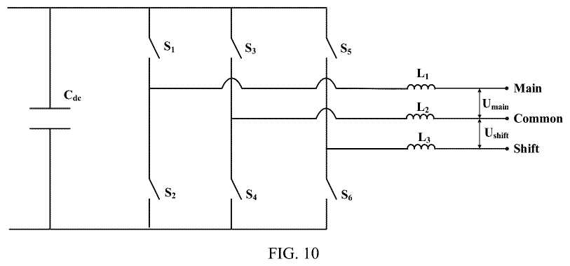

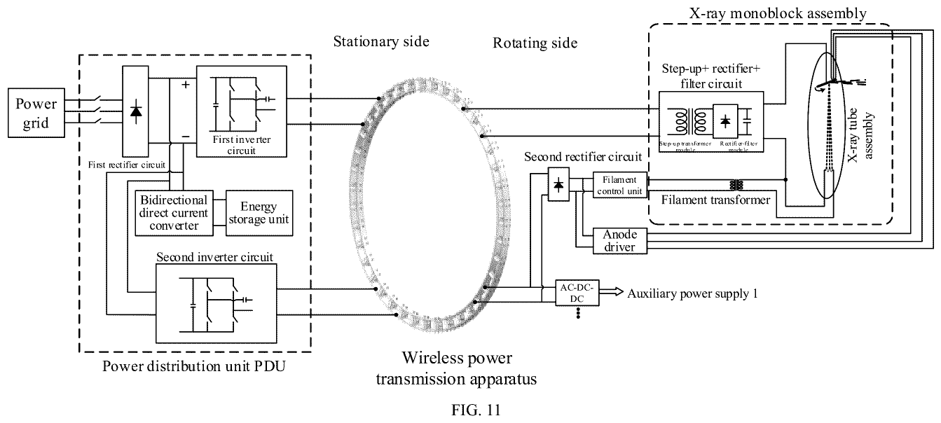

In an optional implementation, as shown in FIG. 10, the imaging device further comprises an anode driver located on the rotating side of the gantry and connected in series between the second rectifier circuit and the X-ray tube assembly. It is used for converting the DC voltage output by the second rectifier circuit into an alternating voltage and loading the alternating voltage to a motor used for driving the anode to rotate in the X-ray tube assembly.

In the example shown in FIG. 10, the anode driver uses a three-phase H-bridge inverter circuit. Three parts of the output of the three-phase H-bridge inverter circuit are connected to a Main end, a Shift end and a Common end, respectively, of a stator winding of the motor in the X-ray tube assembly after passing through filter inductors L1, L2 and L3, respectively, and two voltages Umain and Ushift output by the three-phase H-bridge inverter circuit drive the anode target to rotate, which is beneficial for heat dissipation of the anode target of the X-ray tube assembly. A frequency of the voltage Umain and that of the voltage Ushift are usually 50-180 Hz.

In an optional implementation, the power distribution unit further comprises an energy storage unit. The energy storage unit is connected to the output end of the first rectifier circuit. Where the energy storage unit may be specifically an energy storage battery. The capacity and quantity of the energy storage battery can be specifically selected as required in application. In this implementation, the requirement of the imaging device on the capacity of the power grid can be lowered by introducing the energy storage unit to a direct current bus.

In an optional implementation, as shown in FIG. 11, the first rectifier circuit is an uncontrollable rectifier circuit, and the power distribution unit further comprises a bidirectional direct current converter connected in series between the first rectifier circuit and the energy storage unit. Where the bidirectional direct current converter may also be referred to as a bidirectional DC-DC converter, which is an apparatus capable of achieving current conversion and achieving bidirectional flowing of direct current. An output current may be positive or negative during use, and the generated energy may achieve bidirectional flowing, that is, the energy can flow from an input side to an output side or from an output side to an input side.

In this implementation, the first rectifier circuit is connected to the energy storage unit through the bidirectional direct current converter. The energy storage unit is connected to the first inverter circuit and the second inverter circuit through the bidirectional direct current converter. By controlling the bidirectional direct current converter, what can be achieved is to charge the energy storage unit by using a direct current bus voltage output by the first rectifier circuit or to supply power to the first inverter circuit and the second inverter circuit by using the energy storage unit.

In a specific implementation, when the X-ray imaging device is used to scan a patient, the bidirectional direct current converter may be controlled to maintain the voltage of the direct current bus constant. In this case, the energy storage unit supplies power to the whole imaging device. When the imaging device is in a standby state, the bidirectional direct current converter is controlled to charge the energy storage unit, so as to ensure that the energy storage unit can continuously maintain high-power scanning of the X-ray imaging device, and the power grid only needs to provide average power of the imaging device. Therefore, the requirement of the imaging device on the capacity of the power grid can be lowered.

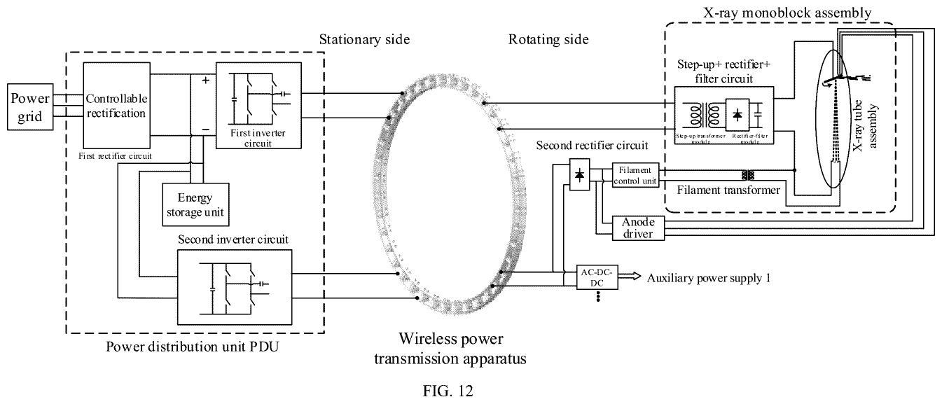

In another optional implementation, as shown in FIG. 12, the first rectifier circuit is a controllable rectifier circuit. By controlling the first rectifier circuit, what can be achieved is to charge the energy storage unit or to supply power to the first inverter circuit and the second inverter circuit by using the energy storage unit.

In a specific implementation, the first rectifier circuit can be controlled to charge the energy storage unit when the imaging device is in a standby state. When the imaging device is used to scan a patient, the first rectifier circuit and the energy storage unit supply power to the first inverter circuit and the second inverter circuit together. In this scheme, the power grid also only needs to provide average power of the imaging device, so that the requirement of the imaging device on the capacity of the power grid can be lowered.

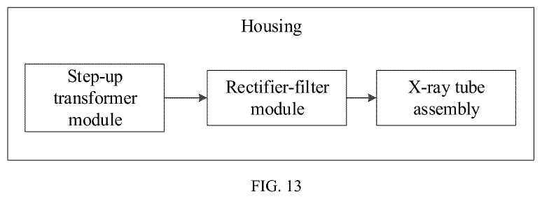

In an optional implementation, as shown in FIG. 13, the step-up transformer module, the rectifier-filter module and the X-ray tube assembly are located in a same housing, and the housing is filled with an insulating medium. Where the insulating medium is usually a liquid dielectric medium for insulation, and may be, for example, transformer oil and the like.

In a specific implementation, a solid insulating material may also be arranged in part inside the housing for insulation and structural supporting. In order to improve the insulation performance between the X-ray tube assembly and the rectifier-filter module, an insulating structure may be arranged between the X-ray tube assembly and the rectifier-filter module.

In this implementation, the step-up transformer module, the rectifier-filter module and the X-ray tube assembly are arranged in the same housing, and the housing is filled with the insulating medium, so that the step-up transformer module, the rectifier-filter module and the X-ray tube assembly can be in the same insulating medium environment. Therefore, an ordinary connector is used between a high-voltage tank comprising the step-up transformer module and the rectifier-filter module and the X-ray tube assembly, without using high-voltage connectors such as a high-voltage plug, a high-voltage socket and a high-voltage cable. This reduces the complexity of high-voltage connection and reduces the costs of high-voltage connection.

In a specific implementation, the step-up transformer module comprises at least two step-up transformers, and the rectifier-filter module comprises at least two rectifier-filter circuits. Primary sides of the step-up transformers are connected in series or in parallel, and secondary sides of the step-up transformers are each connected to an input end of at least one corresponding rectifier-filter circuit. Output ends of all the rectifier-filter circuits are connected in series.

In a specific implementation, the rectifier-filter circuits may be full-bridge rectifier-filter circuits, or may be voltage doubler rectifier-filter circuits, or the like. Where different rectifier-filter circuits can be located on different printed circuit boards (PCBs), and all the PCBs are stacked. Where the PCBs with the rectifier-filter circuits can also be referred to as rectifier plates.

In order to prevent leakage of the X-rays from a periphery of the housing, in an optional implementation, a shielding structure used for shielding the X-rays is arranged on an outer side of a tube core of the X-ray tube assembly, so that a high-voltage circuit can be prevented from being exposed to X-ray radiation for a long time to accelerate the aging and failure of elements. In a specific implementation, a layer of lead may be arranged around the tube core to shield the X-rays.

In an optional implementation, the rectifier plate is in the shape of a circular arc, preferably, in the shape of a ¾ ring. In order to reduce the volume of a transformer as much as possible, the step-up transformer and the filament transformer may be planar transformers.

Although specific embodiments of the present disclosure have been described above, it should be understood by those skilled in the art that these embodiments are merely illustrative and that the protection scope of the present disclosure is defined by the appended claims. Various changes or modifications may be made to these embodiments by those skilled in the art without departing from the principle and spirit of the present disclosure, and such changes and modifications shall fall within the protection scope of the present disclosure.

What is claimed is:

1. A wireless power transmission apparatus, comprising:
a first annular structure, provided with a first magnetic core assembly and a first framework, wherein the first magnetic core assembly is arranged on a circumference of the first annular structure, the first magnetic core assembly comprises at least one magnetic core, the first framework is wound with a first winding, and the first winding passes through a window of the magnetic core in the first magnetic core assembly; and
a second annular structure, provided with a second framework, wherein the second framework is wound with a second winding, and the second winding passes through the window of the magnetic core in the first magnetic core assembly;
wherein a gap is present between the first winding and the second winding, the second annular structure is rotatable relative to the first annular structure, and the first winding is used for receiving an alternating voltage and transmitting the alternating voltage to the second winding.

2. The wireless power transmission apparatus according to claim 1, wherein the magnetic core in the first magnetic core assembly has a magnetic core gap, a size of the magnetic core gap meets a requirement for the second framework to pass through the magnetic core gap, and the second framework passes through the magnetic core gap and is fixed on the second annular structure.

3. The wireless power transmission apparatus according to claim 2, wherein the first magnetic core assembly is arranged on an inner side or an outer side of the circumference of the first annular structure.

4. The wireless power transmission apparatus according to claim 1, wherein the magnetic cores in the first magnetic core assembly are uniformly arranged on the circumference of the first annular structure.

5. The wireless power transmission apparatus according to claim 1, wherein the first framework and/or the second framework is a closed annular structure.

6. The wireless power transmission apparatus according to claim 1, wherein the first frameworks are a plurality of different structures, and/or the second frameworks are a plurality of different structures.

7. The wireless power transmission apparatus according to claim 1, wherein the circumference of the first annular structure is also provided with a second magnetic core assembly, the second magnetic core assembly comprises at least one magnetic core, the first annular structure is also provided with a third framework, the third framework is wound with a third winding, and the third winding passes through a window of the magnetic core in the second magnetic core assembly;
the second annular structure is also provided with a fourth framework, the fourth framework is wound with a fourth winding, and the fourth winding passes through the window of the magnetic core in the second magnetic core assembly;
wherein a gap is present between the third winding and the fourth winding, and the third winding is used for receiving an alternating voltage and transmitting the alternating voltage to the fourth winding.

8. The wireless power transmission apparatus according to claim 7, wherein the magnetic core in the second magnetic core assembly has a magnetic core gap, a size of the magnetic core gap meets a requirement for the fourth framework to pass through the magnetic core gap, and the fourth framework passes through the magnetic core gap and is fixed on the second annular structure.

9. The wireless power transmission apparatus according to claim 7, wherein the magnetic cores in the second magnetic core assembly are uniformly arranged on the circumference of the first annular structure.

10. The wireless power transmission apparatus according to claim 7, wherein the second magnetic core assembly and the first magnetic core assembly are arranged side by side on the circumference of the first annular structure, the third framework and the first framework are arranged side by side on the first annular structure, and the fourth framework and the second framework are arranged side by side on the second annular structure.

11. The wireless power transmission apparatus according to claim 10, wherein the third framework and the first framework are arranged side by side on the inner side or the outer side of the first annular structure.

12. The wireless power transmission apparatus according to claim 7, wherein the third frameworks and the first frameworks are in one-to-one correspondence in position or are arranged in an alternating manner.

13. The wireless power transmission apparatus according to claim 7, wherein the fourth frameworks and the second frameworks are in one-to-one correspondence in position or are arranged in an alternating manner.

14. The wireless power transmission apparatus according claim 7, wherein the quantity of the third frameworks and the quantity of the first frameworks are different, and/or the quantity of the fourth frameworks and the quantity of the second frameworks are different.

15. An imaging device, comprising a gantry and the wireless power transmission apparatus according to claim 1, wherein the first annular structure of the wireless power transmission apparatus is located on a stationary side of the gantry, and the second annular structure of the wireless power transmission apparatus is located on a rotating side of the gantry.

16. The imaging device according to claim 15, wherein the imaging device further comprises a power distribution unit, a step-up transformer module, a rectifier-filter module and an X-ray tube assembly, the power distribution unit is located on the stationary side of the gantry and comprises a first rectifier circuit and a first inverter circuit, and the step-up transformer module, the rectifier-filter module and the X-ray tube assembly are all located on the rotating side of the gantry;
an output end of the first rectifier circuit is connected to an input end of the first inverter circuit, an output end of the first inverter circuit is connected to the first winding in the wireless power transmission apparatus, the second winding in the wireless power transmission apparatus is connected to an input end of the step-up transformer module, an output end of the step-up transformer module is connected to an input end of the rectifier-filter module, and an output end of the rectifier-filter module is connected to the X-ray tube assembly;
the first rectifier circuit is used for receiving an alternating voltage provided by a power grid, rectifying the alternating voltage and outputting a DC voltage;
the first inverter circuit is used for converting the DC voltage output by the first rectifier circuit into an alternating voltage and transmitting the alternating voltage to the step-up transformer module through the first winding and the second winding in the wireless power transmission apparatus;
the step-up transformer module is used for receiving the alternating voltage transmitted by the wireless power transmission apparatus and stepping up the alternating voltage; and
the rectifier-filter module is used for rectifying and filtering the stepped-up alternating voltage and applying an obtained DC voltage to the X-ray tube assembly.

17. The imaging device according to claim 16, wherein the step-up transformer module, the rectifier-filter module and the X-ray tube assembly are located in a same housing, and the housing is filled with an insulating medium.

18. The imaging device according to claim 16, wherein the power distribution unit further comprises an energy storage unit, and the energy storage unit is connected to the output end of the first rectifier circuit.

19. The imaging device according to claim 18, wherein the first rectifier circuit is an uncontrollable rectifier circuit, and the power distribution unit further comprises a bidirectional direct current converter connected in series between the first rectifier circuit and the energy storage unit.

20. The imaging device according to claim 16, wherein the power distribution unit further comprises a second inverter circuit, the imaging device further comprises a second rectifier circuit, a filament transformer and a filament control unit, the second rectifier circuit, the filament transformer and the filament control unit are all located on the rotating side of the gantry, the filament transformer comprises a primary winding and a secondary winding, and the X-ray tube assembly comprises a filament;
the output end of the first rectifier circuit is also connected to an input end of the second inverter circuit, an output end of the second inverter circuit is connected to the third winding in the wireless power transmission apparatus, the fourth winding in the wireless power transmission apparatus is connected to an input end of the second rectifier circuit, an output end of the second rectifier circuit is connected to an input end of the filament control unit, an output end of the filament control unit is connected to the primary winding of the filament transformer, and the secondary winding of the filament transformer is connected to the filament;
the second inverter circuit is used for converting the DC voltage output by the first rectifier circuit into an alternating voltage and transmitting the alternating voltage to the second rectifier circuit through the third winding and the fourth winding in the wireless power transmission apparatus;
the second rectifier circuit is used for receiving the alternating voltage transmitted by the wireless power transmission apparatus and rectifying the alternating voltage to obtain a DC voltage; and
the filament control unit is used for converting the DC voltage output by the second rectifier circuit into an alternating voltage and loading the alternating voltage to the primary winding of the filament transformer.
